(19) United States
(12) Patent Application Publication
(10) Pub. No.: US 2026/0263564 A1
(43) Pub. Date: Sep. 10, 2026

(54) LAMININ GENERATION AND INJECTION METHOD AND SYSTEM

(71) Applicant: PHC, LLC, Silver Spring, MD (US)

(72) Inventors: Luiz C.N. Sampaio, Houston, TX (US); Doris A Taylor, Elmore, AL (US); Patrick Herda, Wilmington, DE (US)

(21) Appl. No.: 19/126,199

(22) PCT Filed: Oct. 31, 2023
(86) PCT No.: PCT/US2023/078284

§ 371 Date: Apr. 30, 2025

Related U.S. Application Data
(60) Provisional application No. 63/420,955, filed on Oct. 31, 2022.

(51) Int. Cl. A61K 38/39; A61K 9/00; A61K 47/02; A61P 9/04; G16H 20/17

(52) U.S. Cl. CPC A61K 38/39; A61K 9/0019; A61K 47/02; A61P 9/04; G16H 20/17

(57) ABSTRACT

A system and method for the production and delivery of biologically active laminin isoform(s) to a target muscle, organ, or system of the body, configured to encourage cell and tissue regeneration and discourage scar formation, facilitating an increase of health of the target system and methods to assess the same.

### CONTINUITY

This application is a non-provisional PCT application of provisional patent application No. 63/420,955, filed on Oct. 31, 2022, and priority is claimed thereto.

### COPYRIGHT NOTICE

A portion of the disclosure of this patent document contains material, which is subject to copyright protection. The copyright owner has no objection to the facsimile reproduction by anyone of the patent document or the patent disclosure, as it appears in the Patent and Trademark Office patent files or records, but otherwise reserves all copyright rights whatsoever.

### FIELD OF THE PRESENT INVENTION

The present invention relates generally to polymerized laminin technology, and more specifically to generating active polymerized laminin and injection (and other delivery) methods and a system for promoting cardiac-based (or other organ/system-based) tissue (re) generation/repair.

### BACKGROUND OF THE PRESENT INVENTION

In Western countries, the overall heart failure (HF) prevalence and incidence are ~2% and 0.2%~, respectively [Savarese & D'Amario, 2018]. About half of the individuals with HF have reduced ejection fraction (HFrEF), and the remaining half either show preserved or mid-range ejection fractions (HFrEF and HFmrEF, respectively). Several cohort studies showed that both the incidence and prevalence of HFpEF increase sharply with age [Dunlay et al., 2017; 2012]. Furthermore, more females have HFpEF whereas more males have HFrEF and HFmrEF. According to a detailed study of left ventricular ejection fraction (EF) in 1,223 patients with HF in Olmsted County, Minnesota, the highest EF for women was ~65% and for men it was ~22%, and these accounted for ~16% and ~12.5% of the patients, respectively [Dunlay et al., 2017; 2012]. Although many treatments have been tested in HFpEF, the European guidelines are categorical: ‘No treatment has yet been shown, convincingly, to reduce morbidity and mortality in patients with HFpEF” (Ferrari, 2015). Clearly, novel interventions are needed for the treatment of HFpEF.

In a healthy heart, cardiac fibroblasts are responsible for maintaining the integrity of the cardiac matrix network and regulating the transmission of mechanical and electrical signals, thus contributing to normal systolic and diastolic function of the ventricle. However, after injury, an inflammatory or reparative response is initiated that effectively debrides dying cells or necrotic tissue in part via macrophages and ultimately heals by fibroblasts converting to myofibroblasts, and forming a collagen-I-based fibrosis. This reactive and disperse fibrosis in the myocardium leads to altered regional biochemical, mechanical and functional properties including alterations to chamber compliance and increased ventricular stiffness, thereby compromising cardiac output. In vitro studies on cardiomyocytes (CM) are frequently performed in a simplified model using a surface coated-plate (2-D cell-based assays). However, cells behave differently, both structurally and functionally, when seeded on 3-D vs. 2-D or when placed in vivo.

The cardiac extra-cellular matrix (ECM) can be described simplistically as a bi-laminar structure with an inner compartment composed of the basement membrane (BM) formed by laminins (LN), collagens (col) IV, XV and XVIII, perlecan, agrin and nidogens 1 and 2 (Farhadian, Contard et al. 1996); and an outer structural compartment, the interstitial matrix, including collagens I, III, VI and XII, proteoglycans, and the associated matricellular proteins (Bowers, Banerjee et al. 2010). These ECM compartments impart mechanical properties (stiffness, anisotropy) to cardiac tissue while orienting the CM in a functional manner. Laminin, a ubiquitous major BM component, including in the heart, plays a pivotal role in cell-matrix interactions.

Laminins (LN), heterotrimeric glycoproteins, are a defining component of all basement membranes (BMs) that polymerize to form network nodes, with a strict requirement to form LN (αβγ) polymers. It was demonstrated that LN self-polymerizes in the absence of any cellular components forming a polymer that recapitulates the in vivo protein architecture called polylaminin (polyLN). The supramolecular organization of polyLN drives its biological function by its interactions with cell receptors (e.g., but not limited to integrin and dystroglycan). This bioactive polymer promotes anti-inflammatory, proangiogenic, and protective effects by modulating immune cell responses, important cardiomyocyte responses, and local cell responses. Laminins have a central role in the formation, architecture, and stability of organs, and they connect the parenchymal cells with the underlying interstitial compartment. The interstitium, a primarily collagenous spatial fluid-containing network, is where mechanical changes are often reflected

Laminins are critical mediators between the cell and the interstitium. If there were a way in which specific isoforms of laminin could be delivered to an unhealthy tissue or organ, healthy biophysical and or biochemical characteristics of the tissue, vessel or organ could theoretically be restored, and parenchymal cell loss could be minimized. Fibrosis is generally a hallmark of virtually every disease, such as vascular fibrosis, or vascular stiffening, or parenchymal scarring. Laminin is an intrinsic connector between cells and the interstitium where these mechanical changes occur. By targeting laminin as the mediator, the method and system of the present invention presents a means by which an organ or tissue may be restored to a healthy capacity.

It is now understood that the basement membrane protein laminin can modulate titin isoform expression in cardiomyocytes, and specifically that in vitro increasing the amount of the laminin a1 isoform present in a cell culture substrate will increase TTN-N2BA isoform expression in cultured human (iPSC-derived) CMs, and that increasing the amount of laminin a2 will increase-N2B expression. Further, there is now evidence that, in human heart samples, the titin isoform ratio associates with the laminin isoform present.

A known technique for protein production is disclosed in U.S. Patent Application Publication No. 2011/0172159 ('159 Pub.). The '159 Pub. describes generating proteic acid polymer generation from the protein laminin diluted in an acidic wash having a divalent cation. The '159 Pub. notes a primary pH of 4.0 but operational pH ranges from 3.0-6.0 and a preferential range of 4.5 to 5.5.

The '159 Pub. provides the general understanding that proteic acid polymers can be used to promote regeneration in various tissue groups, including cardiac muscle. The '159 Pub. fails to provide any disclosure or teaching of how to promote regeneration outside of the exemplary mammal-based spinal cord injury in a period of less than 30 days from lesion occurrence.

Further noted in the prior art is the opinion article “Change the Laminin, Change the Cardiomyocyte: Improve Untreatable Heart Failure” published by the International Journal of Molecular Sciences in 2020 (Laminin Op-Ed). The Laminin Op-Ed indicates the generally acknowledged problems of heart failure with preserved ejection fraction (HFpEF), as well as indicating potential solutions may be found by recognizing and seeking to address cardiomyocyte stiffness or tissue compliance.

The Laminin Op-Ed note that cardiomyocyte stiffness is regulated, in general, by intracellular titin. Laminin is a major component of the extracellular matrix, which makes up the basement membrane having the cardiomyocytes and other cells localized therein. The Laminin Op-Ed indicates preliminary research potentially supporting that the modulating of basement membrane laminin isoforms can alter titin isoform expression in human-induced pluripotent stem cell-derived cardiomyocytes. The Laminin Op-Ed provides that one potential outcome may be for altering cardiac titin isoform ratios to induce structure and functional changes at the cellular level, including potentially altering or increase cardiac compliance.

While the Laminin Op-Ed notes forward-thinking objectives for uses and benefits, it fails to provide any technical disclosure for performing these regenerative or stiffening operations.

The present method and system improves upon the existing prior art knowledge, including improving active laminin manufacturing and operational methodologies for injection (or other delivery mechanism) of laminin proteins for regenerative functions.

### SUMMARY OF THE PRESENT INVENTION

The present invention relates to the use of an active polymerized laminin to promote tissue (re) generation/repair. The pleiotropic effects of this proposed active biopharmaceutical can be especially beneficial in the context of a multifactorial cardiac disease such as HFpEF, HFrEF, and HFmEF and or in other organs chronic fibrotic conditions.

The method and system of the present invention includes both laminin production as well as methodologies for injections (or other delivery mechanisms).

The present method and system provides a solution for the treatment of heart failure with preserved ejection fraction and chronic fibrosis in other organs. The present method and system solve a need, providing therapeutic strategies not previously available. Whereas the conventional treatment currently previously aimed to stop the progression of initial damage, (i.e., it tries to reduce the inflammatory reaction, oxidative stress, and secondary tissue damage, and failing to address a curative or regenerative solution) the active biopharmaceutical of the present invention can induce cardiac tissue repair or regeneration, including in one embodiment via intramyocardial injection(s).

The following brief and detailed descriptions of the drawings are provided to explain possible embodiments of the present invention but are not provided to limit the scope of the present invention as expressed herein this summary section.

### BRIEF DESCRIPTION OF THE DRAWINGS OF THE PRESENT INVENTION

The accompanying drawings, which are incorporated herein and form a part of the specification, illustrate the present invention and, together with the description, further serve to explain the principles of the invention and to enable a person skilled in the pertinent art to make and use the invention.

The present invention will be better understood with reference to the appended drawing sheets, wherein:

FIG. 1 details a schematic view of ECM-cells interaction in an adult non-failing heart, laminin (LN) α2 is predominant in the basement membrane, N2B is the predominant titin (TTN) isoform (via PI3K-RBM20 phosphorylation), and Fibroblasts (FB) are present but inactive. PI3K=Phosphoinositide 3-kinase, RBM20=RNA-binding-Protein 20, col=Colagen, AKT (Protein-kinase-B—PKB), mTORC1=mammalian target of rapamycin complex 1, SRPK=Serine-arginine protein kinases, PIP2=Phosphatidylinositol bi-phosphate, PIP3=Phosphatidyl inositol tri-phosphate.

FIG. 2 details a view of ECM-cells interaction in a failing heart, in HFpEF, where type 1 macrophages (MΦ) increase in number, inflammatory cytokines are secreted, myofibroblasts (MyoFB) are present, and fibrosis and matrix stiffness are generated. An abnormal increase of TTN-N2B synthesis via PI3K signaling leads to a decreased N2BA:N2B ratio, which reduces CM compliance against increased ECM stiffness. TNF-@=Tumor necrosis Factor alpha, MMP=matrix metalloproteinase, cAMP=cyclic Adenosine Mono Phosphate, IL=Interleukin, TGF-β=Transforming growth factor Beta, RIP1=Receptor Interacting Protein kinase1.

FIG. 3 details a view of a heart treated via the system and method of the present invention wherein, in compensated HFpEF or with appropriate corrective Bioactive Laminin Polymers (active biopharmaceutical treatment), cAMP activation alters the response to inflammation, inhibits RBM20 phosphorylation, increases TTN-N2BA production, and activates PKA & PKG to phosphorylate TTN-N2B spring regions. The resulting increased N2BA:N2B ratio with phosphorylated N2B improves CM compliance and compensates diastolic dysfunction. And in noncardiomyocytes, the active biopharmaceutical also promotes a non-inflamed cell phenotype. In Fibroblasts, cAMP inhibits the myofibroblasts phenotype (↓collagen I, TNF-α. and MMP-2/MMP-9 secretion), and in macrophages (MΦ) a shift will occur with a decrease in M1 and an increase of M2 phenotypes. AC=Adenyl cyclase, PKA=Protein Kinase A, Protein Kinase G. ATP=Adenosyl tri-phosphate.

FIG. 4 details a schematic representation of a system for treating chronic diseases by using a laminin-based active biopharmaceutical. The system includes the steps for determining the appropriate laminin isoforms based on the target organ and personalized patient information (e.g. clinical characteristics, diagnostic data, demographics), producing the correct laminin isoform mixture, activating the product, and finally delivering the active biopharmaceutical for treatment.

FIG. 5 depicts laminin Isoform expression during human heart development. During Human development, laminin isoforms change as cardiac function matures. The fetal isoforms (e.g. LN111) predominate during fetal development and decline post-natally. Transient isoforms (e.g. LN511) are expressed early in life while the adult isoforms (e.g. LN211) increase and predominate later.

### DETAILED DESCRIPTION OF THE PREFERRED EMBODIMENTS OF THE PRESENT INVENTION

The present specification discloses one or more embodiments that incorporate the features of the invention. The disclosed embodiment(s) merely exemplify the invention. The scope of the invention is not limited to the disclosed embodiment(s).

References in the specification to “one embodiment,” “an embodiment,” “an example embodiment,” etc., indicate that the embodiment described may include a particular feature, structure, or characteristic, but every embodiment may not necessarily include the particular feature, structure or characteristic. Moreover, such phrases are not necessarily referring to the same embodiment, Further, when a particular feature, structure, or characteristic is described in connection with an embodiment, it is submitted that it is within the knowledge of one skilled in the art to affect such feature, structure, or characteristic in connection with other embodiments whether or not explicitly described.

The present invention is a system and method for the production of active laminin polymers and ultimately the delivery of the active laminin polymers to target areas of a tissue or organ in need of repair and/or regeneration.

Laminin production via the method of the present invention includes the polymerization of laminin in an acidic buffer supplemented with a halide salt. In one embodiment, the acidic buffer is at a pH level of about or at 4.0. In another embodiment, the acidic buffer can be at any pH level below 7.0 and is not expressly required within the range of 4.0 or 3.0 to 6.0 of the '159 Pub. disclosure.

Delivery of the applicable isoform of laminin via injection may occur within standard injection techniques and at multiple temperature ranges, including at or around room-temperature or in various embodiments at lower temperatures. Alternately, delivery may also be accomplished via one or more of the following: 1) In vivo infusion, local gel application, nanoparticles via encapsulation, or viral vector delivery; 2) In vitro via local in vitro culture platform, by overlay of cells or by exposure to powdered substrate; or 3) exposure of cells to laminin prior to cell delivery.

In one embodiment, the calcium supplement includes the halide salt calcium chloride. In one embodiment, the calcium chloride is supplemented at a ratio of or about 1 mM. In further embodiments, the ratio of calcium chloride may be within a varying range above or below the 1 mM.

The present invention may use any suitable alternative input to calcium chloride. For example, one embodiment may use magnesium as a suitable alternative. Other embodiments can use any other suitable supplement material having similar or identical chemical properties, as recognized by one skilled in the art, and the supplement is not limited to calcium chloride.

In one embodiment, the laminin is stored in a frozen state. The stored laminin can be divided into samples or sizing for individual treatments. Therefore, the laminin sample is thawed to a temperature for mixing operations. In one embodiment, the temperature may be on or about room temperature with a standard variance as recognized by one skilled in the art. In another embodiment, the temperature may be a lower temperature aligning with the mixture elements. In another embodiment, the temperature may be a higher temperature facilitating mixture and reactive operations.

In one embodiment, the laminin is stored in a powdered state and is preferably mixed with cell culture media or acidic PBS.

The laminin specimen is preferably mixed with the acidic buffer having the halide salt-based supplement therein. In one embodiment, the mixture of the laminin specimen with the buffer has a concentration of or about 50 ug/ml. It is recognized that the concentration of or about 50 ug/ml is one embodiment and that varying concentrations are within the scope of the present invention, including a concentration lower than or greater than the 50 ug/ml.

The present method and system includes preparation of the mixture for injection into an organ or a cardiac muscle-based system or structure. In one embodiment, the injection can be directly into the heart muscle (or other target muscle-based system/organ) of a patient. Alternately, in other embodiments, the mixture can be infused into the target organ, applied to the target organ or system via a gel or vesicle, delivered via nanoparticles, or delivered via a viral carrying product.

In one embodiment, the preparation of the injection mixture is based on or personalized to recipient conditions, including gender, age, height, weight, mobility. respiratory function, degree of muscle degradation, as well as factors recognized by one skilled in the art.

In one embodiment, an injection volume is determined as a total volume of 100 μL encompassing multiple injections to yield 1 μg/Kg of recipient body weight. Injection may be performed using any suitable or known technique herein listed.

For example, but not limited to, one injection technique may include a catheter injection originating in the groin or the upper limbs of the patient. Injection techniques may also include using known instruments, for example one embodiment may include injection using a needle that can range from 16 G to 31 G in size or an infusion catheter of known size.

In addition to the personalization of injection mixture, injection protocols can also be varied or tailored based on the recipient. For example, it is known that injections and the injected therapies operate different on male patients versus female patients. For example, injection protocols can account for physical characteristics of the patient, such as age, height, weight, degree of muscle degradation, etc. For example, the degree of heart failure can also affect the injection protocol.

In one embodiment, the injection method includes a sequenced operation of injections as defined locations and timing. In one embodiment, the total injection volume is injected into the targeted organ and or all cardiac walls (e.g. but not limited to anterior, lateral, inferior, and septal walls). In this embodiment, injection is performed by injecting individual injections in each wall, for a total injection number that contemplates usage of the total volume of the final solution created based on patient's ideal total dosage. In this embodiment, the injections are performed for solution infusion over a minimum of 20 seconds per injection.

The present method may include any variation of injection volume, injection route, iteration, diffusion rates, and timing between injections and the above embodiment is not expressly limited. For example, one embodiment can include varying sequence of all wall-specific injections prior to moving to the next wall, where other embodiments may include one injection per wall in a circular sequence until all injections are completed, and or even multiple injections in only one wall.

The present method injection includes one or more injections or insertion techniques for delivering the laminin solution into a cardiac muscle at any region or damaged regions of the heart, including muscle, vessels, and valvular structures or the parenchyma/any anatomical region of any target organ. The injection and diffusion of the solution into the cardiac muscle allows for laminin supporting the cellular structure of the cardiac wall or cardiac substructure. The laminin insertion into cardiac muscle region facilitates the cells improving operation including reversing degenerative condition(s).

The method and system include not only the preparation of the laminin solution as noted above but also the injection process, routine, and injection solution/volumes relative to the cardiac muscle, segments (walls) and or any anatomical area of a targeted organ.

It should be noted that protein stocks (100 μg/ml) of human recombinant α1β1γ1 laminin (Biolamina; Sweden cat No. LN111) are kept frozen in working aliquots until being diluted with the buffer, prewarmed to 37° C., immediately before activation and or injection. Laminin is diluted to 50 μg/ml in acidic sodium acetate buffer pH4 containing 1 mM CaCl2 to generate the active laminin complex (active biopharmaceutical). A total volume of 100 μL encompassing 1 μg/kg (of body weight) is injected directly into the parenchyma of the targeted organ, infused over a minimum of 20 seconds.

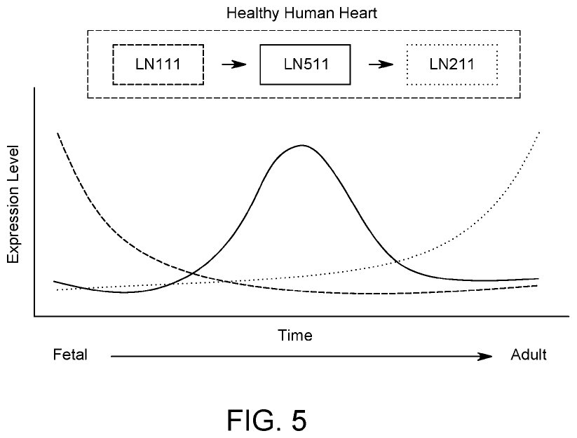

In addition to the above disclosure, laminin and injection protocols provide additional benefits beyond cardiac solutions. The laminin functionality, applicable to cardiomyocytes, can generate similar benefits for other tissue, for example for inflamed or fibrotic tissue in any location. Laminin isoforms change during development, (e.g, FIG. 5) this change is usable in treating diseased tissue. The laminin isoform changes can provide healthy isoforms based on injection(s) as specific locations and types of tissue.

In one embodiment, tissue repair can be asymmetric repair, the tissue repairing from the outside inwards. For example, in the heart, a scar or a portion of a scar may be hibernating myocytes. Laminin insertion using the techniques noted herein can therefore, repair the damaged or diseased tissue, from the border inwards allowing the hibernating cells/heart muscle to contract/pump again.

The present method and system include benefits or uses for the active biopharmaceutical as disclosed herein.

The present invention uses laminin injection therapies for retraining or remodeling cellular functions. In addition to the above embodiment of a cardiomyocyte cells, the chemical and biological reactions based on the laminin injection can operate outside the cardio benefits with a living patient.

For example, the method and system include generation of the active biopharmaceutical as described above. The active biopharmaceutical includes resultant properties including: (1) presenting a regenerative and or anti-inflammatory environment; (2) modulating titin isoform(s) of cardiomyocytes; (3) modulating cardiomyocyte stiffness or compliance; and (4) reducing tissue fibrosis. The biological and operative benefits of the biopharmaceutical are available based on preparation and injection methods noted herein.

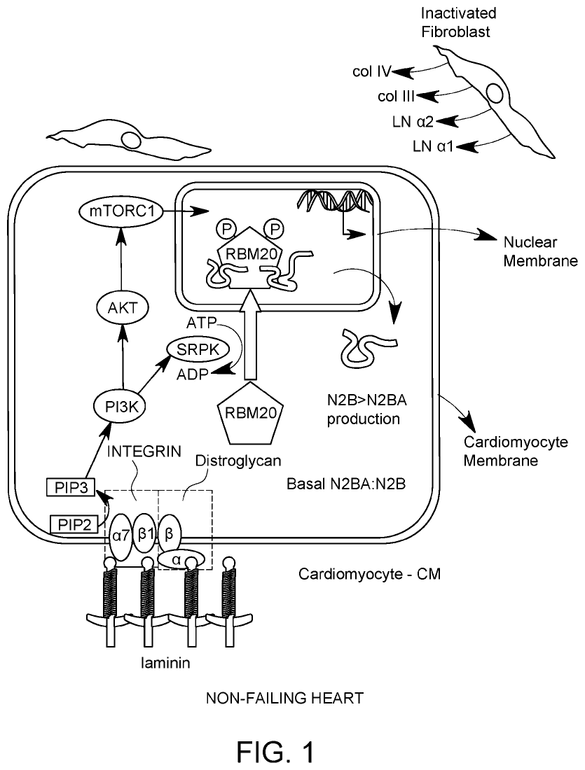

For further illustration, FIG. 1 details a schematic view of ECM-cells interaction in an adult non-failing heart, laminin (LM) α2 is predominant in the basement membrane, N2B is the predominant titin (TTN) isoform (via PI3K-RBM20 phosphorylation), and Fibroblasts (FB) are present but inactive. PI3K=Phosphoinositide 3-kinase, RBM20=RNA-binding-Protein 20, col=Colagen, AKT (Protein-kinase-B—PKB), mTORC1=mammalian target of rapamycin complex 1, SRPK=Serine-arginine protein kinases, PIP2=Phosphatidylinositol bi-phosphate, PIP3=Phosphatidyl inositol tri-phosphate.

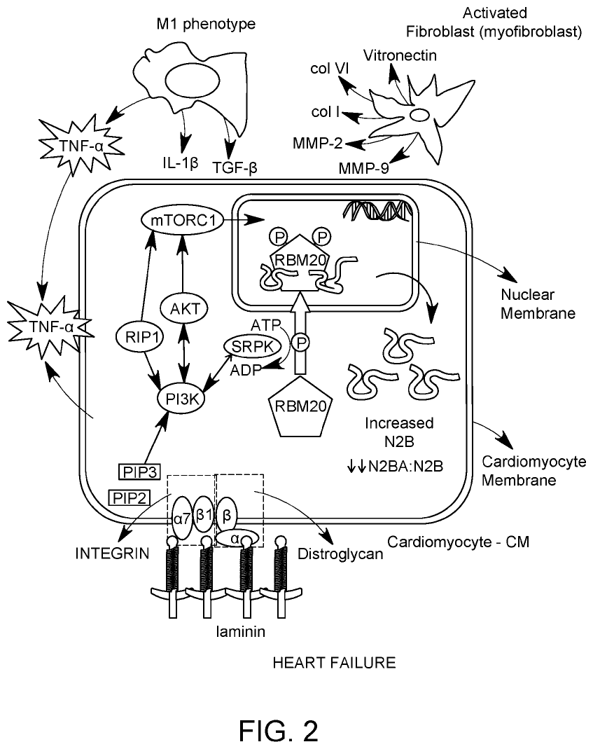

In contrast, FIG. 2 details a view of ECM-cells interaction in a failing heart, in HFpEF, where type 1 macrophages (MΦ) increase in number, inflammatory cytokines are secreted, myofibroblasts (MyoFB) are present, and fibrosis and matrix stiffness are generated. An abnormal increase of TTN-N2B synthesis via PI3K signaling leads to a decreased N2BA:N2B ratio, which reduces CM compliance against increased ECM stiffness. TNF-@=Tumor necrosis Factor alpha, MMP=matrix metalloproteinase, cAMP=cyclic Adenosine Mono Phosphate, IL=Interleukin, TGF-β=Transforming growth factor Beta, RIP1=Receptor Interacting Protein kinase1.

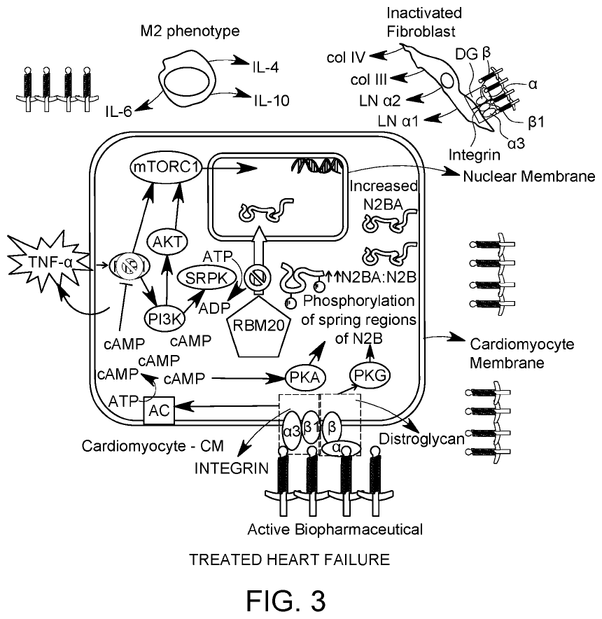

FIG. 3 details a view of ECM-cells interaction in a heart treated via the system and method of the present invention wherein, in compensated HFpEF or with appropriate corrective Bioactive Laminin Polymers (active biopharmaceutical treatment), cAMP activation alters the response to inflammation, inhibits RBM20 phosphorylation, increases TTN-N2BA production, and activates PKA & PKG to phosphorylate TTN-N2B spring regions. The resulting increased N2BA:N2B ratio with phosphorylated N2B improves CM compliance and compensates diastolic dysfunction. And in noncardiomyocytes, the active biopharmaceutical also promotes a non-inflamed cell phenotype. In Fibroblasts, cAMP inhibits the myofibroblasts phenotype (↓collagen I, TNF-α. and MMP-2/MMP-9 secretion), and in macrophages (MΦ) a shift will occur with a decrease in M1 and an increase of M2 phenotypes. AC=Adenyl cyclase, PKA=Protein Kinase A, Protein Kinase G. ATP=Adenosyl tri-phosphate.

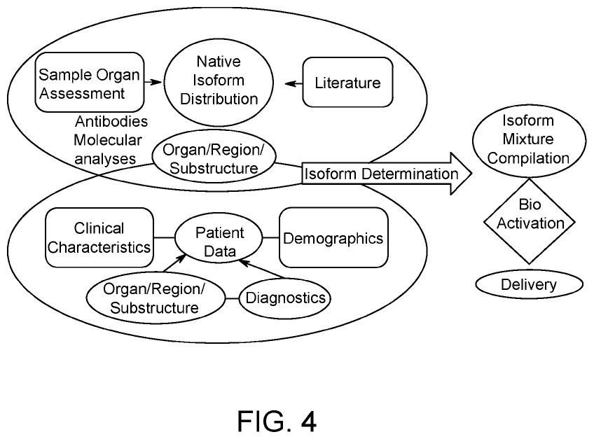

FIG. 4 depicts a schematic representation of a system for treating chronic diseases by using a laminin-based active biopharmaceutical. The system includes the steps for determining the appropriate laminin isoforms based on the target organ and personalized patient information (e.g. clinical characteristics, diagnostic data, demographics), producing the correct laminin isoform mixture, activating the product, and finally delivering the active biopharmaceutical for treatment.

FIG. 5 details laminin isoform expression during human heart development. During Human development, laminin isoforms change as cardiac function matures. The fetal isoforms (e.g. LN111) predominate during fetal development and decline post-nataly. Transient isoforms (e.g. LN511) are expressed early in life while the adult isoforms (e.g. LN211) increase and predominate later.

Additionally, In Fibroblasts, cAMP inhibits the myofibroblasts phenotype (↓collagen I, TNF-α. and MMP-2/MMP-9 secretion), and in macrophages (MΦ) a shift will occur with a decrease in M1 and an increase of M2 phenotypes.

It should be understood that specific isoforms of the laminin polymers are required as they relate to the target organ or tissue (system specific organ, intrinsic structures of an organ), age of the patient, and (potentially) sex and degree of disease of the patient. One example is the key laminin isoforms important for skin and corneal wound healing and re-epithelialization that include, but are not expressly limited to:
LN332 (laminin-5)—Rapidly upregulated, deposited at leading edge to promote re-epithelialization.
LN511—Component of corneal BMs, decreased levels correlate with impaired healing.
LN3A11—Expressed in corneal/skin tissues, may participate in re-epithelialization.
LN3B32 (laminin-5B)—Supports keratinocyte/epithelial cell adhesion, migration, proliferation.
LN521—Expressed in dermis and epidermis, may promote re-epithelialization and angiogenesis.
LN311—May assemble into networks in epithelial BMs for structural support.
LN411 (laminin-8)—Major component of endothelial BMs, promotes angiogenesis.
LN111—Component of epithelial BMs, decreased levels correlated with impaired corneal wound healing.
LN121—Minor component of epithelial BMs.
LN211—Minor component of epithelial BMs.
LN213—Minor component of epithelial BMs.
LN221—Minor component expressed in heart and lung.
LN241—Minor component expressed in heart and lung.
LN411—Minor component in endothelial BMs.
LN421—Minor component in endothelial BMs.
LN423—Minor component in endothelial BMs.
LN511—Also expressed in skeletal and cardiac muscle BMs.
LN521—Also expressed in skeletal and cardiac muscle BMs.
LN524—Minor isoform in neuromuscular junction BMs.

LN332, LN511, LN311, LN332, LN521, LN311 and LN411 seem to be the most critical for skin and corneal wound healing and re-epithelialization.
LN423—Retina-laminin isoform that is specific to the retina
LN523 contains the α5, β2, and γ3 laminin chains.
It is a novel human recombinant laminin isoform generated to recapitulate the retinal interphotoreceptor matrix.
LN523 promotes differentiation of human pluripotent stem cells into photoreceptor progenitors.
Transplantation of LN523-derived photoreceptor progenitors can partially restore retina function in animal models.
The γ3 chain of LN523 is a component of unique CNS laminins and is heavily deposited in retinal blood vessel basement membranes.
Mutations in the laminin γ3 chain can impair retinal vascular development.

LN523 has potential for generating photoreceptor cell therapies to treat retinal degenerative diseases.

It should be noted that the biologically active laminin produced via the system and method of the present invention is not naturally occurring and not commercially available. Further, it should be understood that it is only the activated form of the alpha, beta, and/or gamma forms of laminin are pertinent for use with the system and method of the present invention.

Further, it should be understood that the system and method of the present invention primarily configured to treat organs and systems of the body, rather than tissues. As such, the present invention is envisioned for treatment of acute and chronic disease states, not traumatic acute disease states.

For one embodiment of the present invention, the target is the endogenous repair processes mediated by reparative cells and scar-forming cells, and altering the balance between pro-inflammatory scar formation vs tissue repair (or local repair), where macrophages have polarized in two different ways. The bioactive laminins of the present invention shift focus to the reparative cells, working towards vessel formation and repair of the organ, rather than scar creation. Once the cells of the targeted organ or system are healthy, they start producing the correct form of laminin—the cells change in response to the therapy, and they are no longer reactive (in a healthy way) to the surrounding environment.

Additional data pertaining to laminin presence in different organs, structures and systems are referenced in the below chart:

| Name | Chains | Major Sites of Expression |
|---|---|---|
| Laminin- | α1β1γ1 | Developing Epithelia, Fetal Kidney |
| 111 | | Tubule, Heart, Lung, Liver, Kidney, |
| | | Pancreas, Salivary Glands, Skeletal |
| | | Muscle System and Tendons, Cartilage, |
| | | Bone Marrow, Skin |
| Laminin- | α1β2γ1 | Myotendinous Junctions, Fetal Kidney |
| 121 | | Tubule, Liver, Peripheral Nervous System, |
| | | Salivary Glands, Skeletal Muscle System |
| | | and Tendons, Cartilage |
| Laminin- | α2β1γ1 | All Myogenic Tissues, Adult Adrenal |
| 211 | | Cortex, Mesangial Matrix of Glomerulus, |
| | | Heart, Adult Lung, Liver, Kidney, |
| | | Pancreas, Central Nervous System, |
| | | Peripheral Nervous System, Salivary |
| | | Glands, Ligaments, Skeletal Muscle |
| | | System and Tendons, Bone, Skin |
| Laminin- | α2β1γ3 | Surface of Ciliated Epithelia |
| 213 | | |
| Laminin- | α2β2γ1 | Neuromuscular Junction, Fetal Lung, |
| 221 | | Mesangial Matrix of Glomerulus, Liver, |
| | | Peripheral Nervous System, Ligaments, |
| | | Skeletal Muscle System, and Tendons |
| Laminin- | α3β1γ1 | Epidermis |
| 311 | | |
| Laminin- | α3β2γ1 | Epidermis |
| 321 | | |
| Laminin- | α3β2γ3 | Central Nervous System, Retina |
| 323 | | |
| Laminin- | α3β3γ1 | Skeletal Muscle System and Tendons, |
| 331 | | Bone Marrow |
| Laminin- | α3β3γ2 | Epidermis, Lung, Liver, Skeletal Muscle |
| 332 | | System and Tendons, Bone, Bone Marrow |
| Laminin- | α4β1γ1 | Endothelium, Adipose Tissue, Fetal |
| 411 | | Adrenal Cortex, Heart, Lung, Liver, |
| | | Kidney, Pancreas, Central Nervous |
| | | System, Peripheral Nervous System, |
| | | Ligaments, Bone Marrow, Skin |
| Laminin- | α4β2γ1 | Endothelium, Smooth Muscle, |
| 421 | | Neuromuscular Junction, Mesangial |
| | | Matrix of Glomerulus, Central Nervous |
| | | System, Ligaments, Bone Marrow |
| Laminin- | α4β2γ3 | Central Nervous System, Retina, Hearing |
| 423 | | System |
| Laminin- | α5β1γ1 | Mature Epithelium, Adult Adrenal Cortex, |
| 511 | | Mature Endothelium, Smooth Muscle, |
| | | Heart, Lung, Liver, Kidney, Pancreas, |
| | | Central Nervous System, Peripheral |
| | | Nervous System, Skeletal Muscle System |
| | | and Tendons, Bone, Cartilage, Bone |
| | | Marrow, Skin |
| Laminin- | α5β2γ1 | Mature Epithelium, Adult Adrenal Cortex, |
| 521 | | Mature Endothelium, Smooth Muscle, |
| | | Neuromuscular Junction, Glomerular |
| | | Basement Membrane. Heart, Lung, Liver, |
| | | Kidney, Pancreas, Central Nervous |
| | | System, Peripheral Nervous System, |
| | | Skeletal Muscle System and Tendons, |
| | | Bone, Cartilage, Bone Marrow, Skin |
| Laminin- | α5β2γ3 | Central Nervous System, Retina, Hearing |
| 523 | | System |

In an alternative embodiment of the present invention, wherein the identification, creation, and activation of multiple Human-recombinant laminin (HRL) isoforms for injection into an animal model for antibody generation include the steps of:
Sensitizing an animal model by the introduction of human-recombinant laminin substrates, designed for the stimulation of a specific antibody response;
Extracting the resulting serum or other bodily fluid (e.g. ascites) post-sensitization from the animal model, isolating high specificity and high sensitivity antibodies specific to each human-recombinant laminin substrate introduced;
Employing the isolated antibodies in identifying the presence, and/or relative distribution (e.g., percentage) of each laminin isoform present in the targeted organ or structure, thereby allowing a comprehensive analysis of the laminin isoform distribution within a given healthy or diseased organ system or structure that can further be classified by age, gender or other known identifiers.

In such cases, the active biopharmaceutical production of an organ/region/substructure or system-specific laminin isoform comprises:
Combining an organ/structure sample of differing developmental ages (e.g., fetal through adult) with isolated antibodies against specific laminin isoforms, initially beginning with a specific organ representation set forth in a data table (Laminin Chart), thereby enabling the effective identification of laminin isoform distribution within a sample or any organ or structure;
Then, analyzing the antibody-stained sample to determine and record the quantitative, or percentage, distribution of different laminin isoforms within the targeted organ or structure;
Then, creating a specific organ/structure/region isoform target mixture based on the laminin isoform distribution determined from analysis, thereby accurately reflecting the natural distribution of laminins within the targeted organ or structure;
It should be noted that using a fetal or perinatal isoform is a preferred embodiment for repair, whereas a natural early developmental stage distribution may be appropriate for maintenance.

The included figures are conceptual illustrations allowing for an explanation of the present invention. Notably, the figures and examples above are not meant to limit the scope of the present invention to a single embodiment, as other embodiments are possible by way of interchange of some or all of the described or illustrated elements.

Moreover, where certain elements of the present invention can be partially or fully implemented using known components, only those portions of such known components that are necessary for an understanding of the present invention are described, and detailed descriptions of other portions of such known components are omitted so as not to obscure the invention. In the present specification, an embodiment showing a singular component should not necessarily be limited to other embodiments including a plurality of the same component, and vice-versa, unless explicitly stated otherwise herein. Further, the present invention encompasses present and future known equivalents to the known components referred to herein by way of illustration.

The foregoing description of the specific embodiments so fully reveals the general nature of the invention that others can, by applying knowledge within the skill of the relevant art(s) (including the contents of the documents cited and incorporated by reference herein), readily modify and/or adapt for various applications such specific embodiments, without undue experimentation, without departing from the general concept of the present invention. Such adaptations and modifications are therefore intended to be within the meaning and range of equivalents of the disclosed embodiments, based on the teaching and guidance presented herein.

We claim:

1. A method for producing and delivering applicable bio-active isoform(s) of laminin(s) to a specific organ or body system target of a recipient comprising:
identifying applicable isoform(s) of laminin(s) as relevant to the organ or body system target;
producing the applicable isoform(s) of laminin(s) in a non-native environment;
biologically activating the laminin(s), forming a mixture of bioactive laminins;
delivering the mixture of bioactive laminin(s) to the organ or body system target; and
wherein relevant delivery of the mixture of bioactive laminin(s) is targeted locally, regionally, and systemically.

2. The method of claim 1, wherein bioactive laminin production includes at least the polymerization of laminin(s) present in the mixture in an acidic buffer supplemented with halide salts.

3. The method of claim 2, wherein the acidic buffer is at a pH level of about or at 4.0.

4. The method of claim 2, wherein the acidic buffer is at any pH level below 7.0 and is not expressly required within the range of 4.0, or 3.0 to 6.0.

5. The method of claim 1, wherein delivery of the bioactive mixture of laminin(s) to the organ or body system target is accomplished via at least one of the following: injection, in vivo infusion, local gel application, nanoparticles via encapsulation, and viral vector delivery.

6. The method of claim 1, wherein delivery of the bioactive mixture of laminin(s) to the organ or body system target is accomplished via at least one of the following: in vitro via local in vitro culture platform(s), by overlay of cells, and by exposure of cells to laminin(s) prior to cell delivery; and
wherein the bioactive mixture of laminin(s) is personalized to recipient conditions, including gender, age, height, weight, mobility. respiratory function, degree or muscle degradation.

7. The method of claim 3, wherein the halide salt supplement includes calcium chloride.

8. The method of claim 3, wherein the mixture of the laminin(s) with the acidic buffer has a concentration of or about 50 μg/ml.

9. The method of claim 2, wherein the mixture is personalized to recipient conditions, including age, height, weight, mobility. respiratory function, degree of muscle degradation.

10. The method for treating chronic fibrosis, wherein biologically active laminin(s) polymers activate localized tissue repair mechanisms by modulating immune cells, fibroblastic cells, and parenchymal cells responses.

11. A method for treating chronic fibrosis (e.g. HFpEF) using an active extracellular matrix protein(s) (ECMP) treatment, said method comprising:
activating cAMP;
inhibiting RBM20 phosphorylation;
increasing the production of TTN-N2BA;
activating PKA & PKG to phosphorylate TTN-N2B spring regions; and
thereby increasing CM compliance to effectively treat diastolic dysfunction.

12. The method of claim 10, wherein biologically active laminin polymers exercise anti-inflammatory, pro-regenerative, and anti-fibrotic effects across specific organ or body system targets, thereby promoting the overall restoration and improvement of the targeted organs and tissues.

13. The method of claim 10, wherein laminin isoforms are matched to target tissues based on the native laminin composition of each tissue; and
wherein matching appropriate laminin isoforms to target organs and tissues enables tissue-specific repair and regeneration.

14. The method of claim 10 wherein the target organs and tissues comprise heart, blood vessels, liver, kidney, skin, cornea, brain, retina, pancreas, intestine, lung, nervous system, neuromuscular junction, skeletal muscle, smooth muscle, cartilage, bone, and adipose tissue.

15. The method of claim 10 wherein, the laminin isoforms comprise at least one of the following: laminin-111, laminin-211, laminin-121, laminin-221, laminin-332, laminin-311, laminin-321, laminin-331, laminin-411, laminin-421, laminin-511, laminin-521, laminin-213, laminin-323, laminin-423, and laminin-523 matched to target tissues.

16. The method of claim 11, wherein the bioactive laminin(s), upon delivery, promote muscle relaxation, especially in muscle tissues, predominantly via interaction with laminin-titin complexes, as exemplified by its action in the heart.

17. The method of claim 10, wherein the injection techniques can be performed systemically, regionally, and locally and may include Direct Myocardial Injection, Catheter-based Intracoronary Delivery, Trans-endocardial Injection via Electromagnetic Navigation, Trans-epicardial Injection during open surgical procedure or Video-Assisted Thoracoscopic Surgery (VATS), Pericardial Delivery, Ultrasound-guided Injection, balloon coupled delivery, peripheral and central venous or ductal injection, lymphatic injection and any body cavity, duct, or anatomical tissue and space; and
wherein injection includes methodologies selected from the group: infusion, deposition, and distribution.

18. A method for treating, and assessing disease states comprising:
injecting an active biopharmaceutical into specific regions of a body structure using a catheter-driven system;
altering the biochemical and/or biophysical properties of the body structure to contain target appropriate pro-reparative ECM utilizing a range of electrical, biomechanical, imaging, and biochemical sensors to gather data on various physical, mechanical and biochemical conditions in the organ and to characterize the tissue; and
analyzing the gathered data using AI algorithms to gauge the effectiveness of the treatment, and to ascertain disease-associated characteristics prior to treatment.

19. The method of claim 18, wherein the injection techniques can be performed systemically, regionally and locally;
wherein the injection techniques are selected from the following group: Direct Myocardial Injection, Catheter-based Intracoronary Delivery, Trans-endocardial Injection via Electromagnetic Navigation, Trans-epicardial Injection during open surgical procedure or Video-Assisted Thoracoscopic Surgery (VATS), Pericardial Delivery, Ultrasound-guided Injection, balloon coupled delivery, peripheral and central venous or ductal injection, lymphatic injection and any body cavity, duct, or anatomical tissue and space; and
wherein injection includes methodologies selected from the group: infusion, deposition, and distribution

20. The method of claim 18, wherein the sensors may include at least one but is not limited to the following: thermocouples, infrared thermal imaging, pressure sensors, microelectrode arrays, impedance sensors, pH sensors, oxygen sensors, ion-selective electrodes, photoplethysmography, ultrasound transducers, accelerometers, strain gauges, RNA sampling, and sequencing tools, magnetic resonance, turbidity sensors, electromagnetic field detectors, gas chromatography sensors, biosensors, humidity sensors, viscometers, and sensors for monitoring contractions, electrical activity, fluid levels, specific ions, glucose, lactate, oxygen levels, genetic regulation and expression patterns, electromagnetic field changes, specific gases, specific molecules, proteins, cytokines, biochemicals, cellular events, moisture levels, specific biochemical reactions and tissue viscosity.
